(19) United States
(12) Patent Application Publication
(10) Pub. No.: US 2015/0166497 A1
(43) Pub. Date: Jun. 18, 2015

(54) CONTROLLED PARTIAL DEPOLYMERIZATION PROCESS FOR POLYTETRAMETHYLENE ETHER GLYCOL MOLECULAR WEIGHT DISTRIBUTION NARROWING

(71) Applicant: INVISTA NORTH AMERICA S.A.R.L., Wilmington, DE (US)

(72) Inventors: Suri N. Dorai, Weston Lakes, TX (US); Qun Sun, Wilmington, DE (US)

(73) Assignee: INVISTA NORTH AMERICA S.A.R.L., Wilmington, DE (US)

(21) Appl. No.: 14/407,637

(22) PCT Filed: Jun. 14, 2013
(86) PCT No.: PCT/US13/45786

§ 371 Date: Dec. 12, 2014

Related U.S. Application Data
(60) Provisional application No. 61/663,835, filed on Jun. 25, 2012.

(51) Int. Cl. C07D 307/08

(52) U.S. Cl. CPC C07D 307/08

(57) ABSTRACT

The present invention provides an improved process for preparing a narrow molecular weight distributed polytetramethylene ether glycol (PTMEG). The product polytetramethylene ether glycol has an increased number average molecular weight, a lowered polydispersity and a reduced molecular weight ratio when compared to the polytetramethylene ether glycol in the corresponding feedstock.

### CROSS-REFERENCE TO RELATED APPLICATION

This application claims benefit of priority from U.S. Provisional Application No. 61/663,835 filed Jun. 25, 2012. This application hereby incorporates by reference this application in its entirety.

### FIELD OF THE INVENTION

The present invention relates to an improved process for treating polytetramethylene ether glycol to prepare a commercially desirable product. More particularly, the invention relates to a highly efficient, cost effective process for preparing a polytetramethylene ether glycol product with a desired number average molecular weight, polydispersity, and molecular weight ratio (MWR) range.

### BACKGROUND OF THE INVENTION

Polytetramethylene ether glycol (PTMEG) is well known for use as soft segments in polyurethanes and other elastomers. This homopolymer is a valuable commodity in the chemical industry, where it is widely used to form segmented copolymers with poly-functional urethanes and polyesters. PTMEG has been shown to impart superior dynamic properties to polyurethane elastomers and fibers.

During the preparation of polyether polyols, such as PTMEG, via the polymerization of tetrahydrofuran (THF) in which acetic acid and acetic anhydride are used, the intermediate products will contain acetate or other end groups. It is well known in the art that these acetate or other end groups must be subsequently converted to the hydroxyl functionality prior to ultimate use. For example, U.S. Pat. No. 4,163,115 discloses the polymerization of THF and/or THF with comonomers to polytetramethylene ether diester using a fluorinated resin catalyst containing sulfonic acid groups, in which the molecular weight is regulated by the addition of an acylium ion precursor to the reaction medium. The patent discloses the use of acetic anhydride and acetic acid in combination with the solid acid catalyst. The polymeric product is isolated by stripping off the unreacted THF and acetic acid/acetic anhydride. The isolated product is the diacetate of polymerized tetrahydrofuran (PTMEA) which must be converted to the corresponding dihydroxy product, polytetramethylene ether glycol (PTMEG). Consequently, the ester end-capped polytetramethylene ether is reacted with and an alkanol such as methanol using a basic catalyst to provide the final product polytetramethylene ether glycol and methyl acetate as a by-product.

U.S. Pat. Nos. 4,230,892 and 4,584,414 disclose processes for the conversion of PTMEA to PTMEG comprising mixing a polytetramethylene ether diester with an alkanol of 1 to 4 carbons, and a catalyst which is an oxide, hydroxide, or alkoxide of an alkaline earth metal or an alkali metal hydroxide or alkoxide, bringing the mixture to its boiling point and holding it there while the vapors of the alkanol/alkyl ester azeotrope which form are continuously removed from the reaction zone, until conversion is essentially complete; and then removing the catalyst.

U.S. Pat. No. 5,852,218 discloses reactive distillation wherein a diester of polyether polyol, e.g. PTMEA, is fed to the top portion of the distillation column along with an effective amount of at least one alkali metal or alkaline earth metal oxide, hydroxide or alkoxide catalyst (e.g., sodium methoxide) and with a C₁ to C₄ alkanol (e.g., methanol) while simultaneously adding to the bottom of the reactive distillation column hot alkanol vapor to sweep any alkanol ester formed by alkanolysis of the diester of polyether polyol upwardly.

Various methods for narrowing the molecular weight distribution of PTMEG have been disclosed in U.S. Pat. Nos. 3,925,484, 5,053,553, 5,282,929, 6,355,846 and 8,138,283. U.S. Pat. No. 3,925,484 discloses a process involving partially depolymerizing PTMEG at a temperature of 120 to 150° C. in the presence of a cross-linked ion exchange resin. U.S. Pat. No. 5,053,553 discloses narrowing the molecular weight distribution of PTMEG in a process involving liquid-liquid extraction with methanol, water and non-polar solvent. U.S. Pat. No. 5,282,929 discloses narrowing the molecular weight distribution of PTMEG in a process involving using at least one short path distillation evaporator apparatus. U.S. Pat. No. 6,355,846 discloses feeding PTMEG and an inert solvent to a stripping apparatus and stripping at a high temperature of 150 to 220° C. to narrow the molecular weight distribution of the PTMEG. U.S. Pat. No. 8,138,283 discloses a method for changing the mean molecular weight of PTMEG during a continuous THF polymerization process involving sampling and adjusting during the process, and at least partly depolymerizing product at high temperature over an acidic catalyst, exemplified as bentonite at a temperature of 180 to 200° C., aluminum oxide at 190 to 210° C., silicon dioxide at 190 to 210° C., ion exchanger Amberlyst XN at 130° C., and tungstophosphoric acid at 150 to 160° C.

It is widely accepted that depolymerization methods can be carried out under different process conditions to obtaining strikingly different results. For example, contacting a feed with a catalyst in a fixed bed reactor provides different results from contacting the same feed and catalyst in a different reactor (i.e.: a batch tank reactor vs. a CSTR or a moving-bed reactor). Similarly, selection of temperature and pressure parameters can be very important in obtaining different degrees of molecular weight narrowing.

Therefore, it would be desirable to provide a process for a PTMEG product without requiring complicated, costly or capital intensive unit operations, such as extraction facilities, short path distillation apparatus, stripping means and the requirement for constant sampling and process adjustment during polymerization of THF.

In addition, it would also be desirable to provide an improved process for treating a PTMEG feedstock to recover a PTMEG product having an increased number average molecular weight, a lowered polydispersity and a reduced molecular weight ratio range.

### SUMMARY OF THE INVENTION

The present invention provides an improved process for treating PTMEG to prepare a commercially desirable product. More particularly, the invention relates to a simple, highly efficient, cost effective process with improved capital productivity for preparing a narrow-molecular-weight-distributed PTMEG product having an increased number average molecular weight, a lowered polydispersity, and a lower molecular weight ratio range, when compared to PTMEG feedstock having a normal number average molecular weight, polydispersity, and molecular weight ratio range.

In an embodiment of the current invention, process for preparing polytetramethylene ether glycol comprises the steps of:

(a) contacting a feedstock comprising polytetramethylene ether glycol with an acid ion exchange resin in a contact vessel for a contact time that is sufficient to partially depolymerize the polytetramethylene ether glycol, wherein the acid ion exchange resin has a concentration of acid sites that is greater than about 5 eq/kg, a particle size that is greater than about 0.3 mm, a surface area in the range from about 25 to about 50 m²/gram, and a pore volume in the range from about 0.15 to about 0.35 cc/gram, and wherein the contact vessel has a temperature in the range from about 50 to about 120° C. and a pressure in the range from about 0 to about 30 psig; and

(b) recovering a product stream from the contact vessel comprising a polytetramethylene ether glycol product, wherein the polytetramethylene ether glycol product has an increased number average molecular weight, a lowered polydispersity and a lower molecular weight ratio when compared to the polytetramethylene ether glycol in the feedstock.

In another embodiment of the current invention, the contact time is in the range from about 5 minutes to about 200 minutes.

In another embodiment of the current invention, the contact time is in the range from about 5 minutes to about 60 minutes.

In another embodiment of the current invention, the acid ion exchange resin loses at least a portion of its catalytic properties at temperature above 130° C.

In another embodiment of the current invention, the acid ion exchange resin loses at least a portion of its catalytic properties at temperature above 150° C.

In another embodiment of the current invention, the contact vessel is a continuous stirred tank reactor.

In another embodiment of the current invention, the contact vessel is a fixed bed reactor.

In another embodiment of the current invention, the temperature is in the range from about 60 to about 100° C. and the pressure is atmospheric pressure.

In another embodiment of the current invention, the temperature is in the range from about 60 to about 100° C. and pressure is atmospheric pressure.

In another embodiment of the current invention, the polytetramethylene ether glycol in the feedstock contains less than 1% by weight of polytetrahydrofuran or tetrahydrofuran copolymers.

In another embodiment of the current invention, the polytetramethylene ether glycol in the feedstock contains less than 1% by weight of esters of polytetrahydrofuran or tetrahydrofuran copolymer.

In another embodiment of the current invention, the process further comprises recovering an effluent comprising tetrahydrofuran and water from the contact vessel.

In another embodiment of the current invention, the polytetramethylene ether glycol in the feedstock has a number average molecular weight in the range from about 400 to about 3,000 dalton, a polydispersity in the range from about 1.9 to about 2.3 and a molecular weight ratio in the range from about 2.2 to about 2.5, and wherein the polytetramethylene ether glycol product has a number average molecular weight in the range from about 400 to about 4,000 dalton, a polydispersity in the range from about 1.2 to about 1.8, and a molecular weight ratio in the range from about 1.7 to about 2.15.

In another embodiment of the current invention, the polytetramethylene ether glycol in the feedstock is obtained from the process to covert a diacetate of polymerized tetrahydrofuran to polytetramethylene ether glycol.

### DETAILED DESCRIPTION OF THE INVENTION

Disclosed herein is an improved process for treating PTMEG to prepare a commercially desirable product. More particularly, the invention relates to a simple, highly efficient, cost effective process with improved capital productivity for preparing a narrow-molecular-weight-distributed PTMEG product having an increased number average molecular weight, a lowered polydispersity, and a lower molecular weight ratio range, when compared to PTMEG feedstock having a normal number average molecular weight, polydispersity, and molecular weight ratio range.

As a result of extensive experimentation, the applicants have unexpectedly discovered an improved process to manufacture commercially desirable PTMEG having an increased number average molecular weight, a lowered polydispersity and a lower molecular weight ratio in a simple, efficient and cost effective manner.

All patents, patent applications, test procedures, priority documents, articles, publications, manuals, and other documents cited herein are fully incorporated by reference to the extent such disclosure is not inconsistent with this invention and for all jurisdictions in which such incorporation is permitted.

The term “polymerization”, as used herein, unless otherwise indicated, includes the term “copolymerization” within its meaning.

The term “PTMEG”, as used herein, unless otherwise indicated, means polytetramethylene ether glycol. PTMEG is also known as polyoxybutylene glycol.

The term “THF”, as used herein, unless otherwise indicated, means tetrahydrofuran and includes within its meaning alkyl substituted tetrahydrofuran capable of copolymerizing with THF, for example 2-methyltetrahydrofuran, 3-methyltetrahydrofuran, and 3-ethyltetrahydrofuran.

The term “polydispersity”, as used herein, means the weight average molecular weight (M_{w}) divided by the number average molecular weight (Mₙ), or M_{w}/Mₙ.

The term “Molecular Weight Ratio” or “MWR”, as used herein, is a measure of broadness of molecular weight distribution. MWR is defined by the empirical equation based on the number average molecular weight (Mₙ) and the melt viscosity average molecular weight (Mᵥ) where MWR=Mᵥ/Mₙ. Mᵥ=antilog (0.493 log η+3.5576), wherein the viscosity η of the PTMEG is in Pa·s at 40° C. Mₙ is determined by titration as in ASTM 222A. A normal PTMEG product usually has an MWR not exceeding 2.07 while a value of 2.0 is generally considered normal.

The term “telogen”, as used herein, means a molecular weight regulating agent. Non-limiting examples of telogens include acetic anhydride as taught in U.S. Pat. No. 8,183,283.

In a method for manufacturing PTMEG, THF can be polymerized using a solid acid catalyst, a telogen a, such as acetic anhydride, and a molecular weight moderator, such as acetic acid, as described in U.S. Pat. No. 4,163,115. Typically the THF conversion to polymer ranges from about 20 to 40% at temperatures of about 40° C. to 50° C. The polymeric product is preferably isolated by stripping off the unreacted THF and acetic acid/acetic anhydride for recycle. The product that is isolated is the polymerized diacetate of tetrahydrofuran (PTMEA), which must be converted to the dihydroxy product polytetramethylene ether glycol (PTMEG) to find application as a raw material in most urethane end use applications.

Typically the products of the initial polymerization process are in the form of acetates (or similar terminal ester groups) which are converted to the hydroxyl group terminated glycols by reacting them with methanol in the presence of transesterification/alkanolysis catalysts. This reaction requires a catalyst to attain reasonable rates. Common methanolysis catalysts useful for this purpose include sodium methoxide (NaOMe or NaOCH₃), sodium hydroxide (NaOH), and calcium oxide. In principle, the catalyst useful for such a reaction is a highly alkaline alkanolysis catalyst generally categorized as an alkali metal or alkaline earth metal oxide, hydroxide or alkoxide catalyst and mixtures thereof as taught in U.S. Pat. Nos. 4,230,892 and 4,584,414. Also useful are alkanolysis catalysts that inherently have some water scavenging capability without loss of catalyst activity (e.g., NaOCH₃/Na₂O system wherein trace water is converted to the catalytically active NaOH). The reaction rate using NaOH/NaOCH₃ is rapid even at room temperature and therefore methanolysis is ordinarily carried out at atmospheric pressure. The by-product in this methanolysis is methyl acetate which forms a lower boiling azeotrope with methanol. The alkanolysis reaction is reversible and therefore continuous removal of methyl acetate is essential to obtain a commercially reasonable conversion rate. In the process taught in U.S. Pat. No. 5,852,218, this is done in a reactive distillation column wherein methanol vapor is fed into the column bottom to strip the polymer of methyl acetate. By stripping methyl acetate in this manner, high conversion of PTMEA to PTMEG, for example greater than 99.999%, is achieved in the column. In contrast to the reactive distillation process at least five sequential continuously stirred reactor stages may be required to achieve a conversion of 99.95% only.

The number average molecular weight of PTMEG, determined by end group analysis using spectroscopic methods well known in the art, can be as high as about 30,000 dalton. The number average molecular weight will usually be in the range from about 400 to about 5,000 dalton, and more commonly will range from about 400 to 3,000 dalton. The product mixture of an alkanolysis process will commonly comprise from about 50 to about 80 wt. % PTMEG and from about 20 to about 50 wt. % alkanol, e.g. methanol.

The process of the present may be carried out in suitable contact vessels known to those skilled in the art. Suitable vessels include a continuous stirred tank reactor, a batch vessel, a fixed bed reactor or any combination of one or more vessel configurations known to those skilled in this art.

The preferred contact vessel for use in the present process comprises a fixed bed, e.g. plug flow, reactor since selectivity is improved with this type vessel. The term “selectivity”, as used herein, is the preferential depolymerization of lower molecular weight species in the feed stock, while minimizing the inzipping of higher molecular weight fractions which will maximize the yield of narrow molecular weight distributed PTMEG with reduced MWR.

This selectivity provides the best commercial product with the highest yield. A back-mix reactor (for example a continuous stirred-tank reactor) may also be used when a greater amount of resin is employed.

In an exemplary embodiment of the present invention, the process is performed in a continuous stirred tank by providing a feedstock comprising PTMEG having less than about 1% by weight of polytetrahydrofuran or tetrahydrofuran copolymers or less than about 1% by weight of esters of polytetrahydrofuran or tetrahydrofuran copolymer. The feedstock is then contacted with an acid ion exchange resin at a temperature in the range from about 50 to about 120° C. and a pressure in the range from about 0 to about 30 psig. In another embodiment of the current invention, the temperature may be in the range from about 60 to about 100° C. and at about normal atmospheric pressure. The contact time in the vessel is sufficient to partially depolymerize the PTMEG at those conditions. In exemplary embodiment of the current invention, the contact time may be from about 5 to about 200 minutes. In another embodiment of the current invention, the contact time may be from about 5 to about 60 minutes. The process of the current invention comprises the step of recovering a polytetramethylene ether glycol product from the contact vessel having an increased number average molecular weight, a lowered polydispersity and a reduced molecular weight ratio when compared to the polytetramethylene ether glycol in the feedstock.

In another embodiment of the current invention, the polytetramethylene ether glycol in the feedstock has a number average molecular weight in the range from about 400 to about 3,000 dalton, a polydispersity in the range from about 1.9 to about 2.3 and a molecular weight ratio in the range from about 2.2 to about 2.5, and wherein the polytetramethylene ether glycol product has a number average molecular weight in the range from about 400 to about 4,000 dalton, a polydispersity in the range from about 1.2 to about 1.8, and a molecular weight ratio in the range from about 1.7 to about 2.15.

In another embodiment of the current invention, the process further comprises recovering an effluent comprising tetrahydrofuran and water from the contact vessel.

The acid ion exchange resin for use herein has a concentration of acid sites that is greater than about 5 eq/kg, a particle size that is greater than about 0.3 mm, a surface area in the range from about 25 to about 50 m²/gram, and a pore volume in the range from about 0.15 to about 0.35 cc/gram. This ion exchange resin loses at least a portion (at least about 2-5%) of its catalytic properties at temperature above 130° C. In another embodiment of the current invention, the ion exchange resin loses at least a portion of its catalytic activity above 150° C.

Suitable acid ion exchange resins include acidic, sulfonic acid, macroreticular polymeric resins based on crosslinked styrene divinylbenzene copolymers. Other suitable acid ion exchange resins include those sold by The Dow Chemical Company under the trademarks Amberlyst® 15, Amberlyst® 35, Amberlyst® 36, and combinations thereof.

The following Examples demonstrate the present invention and its capability for use. The invention is capable of other and different embodiments, and its several details are capable of modifications in various apparent respects, without departing from the spirit and scope of the present invention. Accordingly, the Examples are to be regarded as illustrative in nature and non-limiting.

### EXAMPLES

In the following examples, each INVISTA's Terathane® PTMEG is used. Amberlyst®-36, a strong acid ion exchange resin, is sold by The Dow Chemical Company.

Each PTMEG sample was treated in a fixed bed stainless steel column of 1″ diameter and 12″ height, which was packed with 79.6 grams Amberlyst®-36 resin to partially depolymerize the PTMEG under mild conditions of temperature from 60-120° C. and pressure below 30 psig, with residence time below 1 hour, and a feed rate of from 2.7 to 8.1 grams/minute. For convenience, the MWR was determined for the examples. In each experiment, the level of depolymerization of the feedstock PTMEG was determined by oven drying the resulting product PTMEG to remove volatiles (THF and water) in a vacuum oven maintained at about 20″ Hg vacuum and 130° C. for 3 hours. In each experiment, the PTMEG was fed through the packed resin bed for three hours and the sample collected from the 2^{nd}-3^{rd} hour was processed for level of de-polymerization in weight % and measured for Mₙ and MWR changes.

### Examples 1-5

Examples 1-5 used PTMEG feed with Mₙ and MWR of 794 g/mol and 2.43, respectively. The PTMEG was pre-warmed in a jacketed feed tank that was kept at 70° C. and fed through the packed resin bed with up flow using a PMI pump. The packed resin bed column jacket temperature was maintained at about 105° C. The bed temperature was measured by a thermocouple that was imbedded inside the resin. Results are shown in Table 1 for various feed rates.

TABLE 1

| | | Bed | Depoly- | | |
|---|---|---|---|---|---|
| Example | Feed Rate, | Temperature, | merization, | Mₙ, | |
| # | g/minute | ° C. | Wt. % | g/mol | MWR |
| Feed | | | 0 | 794 | 2.43 |
| 1 | 2.7 | 95.3 | 22.4 | 1167 | 1.79 |
| 2 | 4.0 | 94.7 | 15.3 | 1036 | 1.94 |
| 3 | 5.4 | 94.2 | 13.4 | 995 | 1.99 |
| 4 | 6.8 | 92.4 | 11.7 | 935 | 2.09 |
| 5 | 8.1 | 90.7 | 9.8 | 899 | 2.15 |

### Example 6-9

In the same packed resin bed column as in Examples 1-5 was fed PTMEG which had a Mₙ and MWR of 1554 g/mol and 2.28, respectively. Instead of varying the feed rate as in Examples 1-5, these experiments were run with different bed temperatures while the feed rate was kept constant at 8.1 g/minute. Results are shown in Table 2.

TABLE 2

| | | Bed | Depoly- | | |
|---|---|---|---|---|---|
| Example | Feed Rate, | Temperature, | merization, | Mₙ, | |
| # | g/minute | ° C. | Wt. % | g/mol | MWR |
| Feed | | | 0 | 1554 | 2.28 |
| 6 | 8.1 | 85.1 | 8.0 | 1886 | 1.96 |
| 7 | 8.1 | 79.3 | 5.8 | 1818 | 2.01 |
| 8 | 8.1 | 74.6 | 5.2 | 1760 | 1.99 |
| 9 | 8.1 | 70.6 | 4.3 | 1730 | 2.09 |

The data from Examples 1-9 presented in Tables 1 and 2 demonstrate that the present improved process provides PTMEG having increased number average Mₙ and a lower MWR than the corresponding feed PTMEG. PTMEG with narrower molecular weight distribution or reduced MWR is preferred for many end user applications.

While the illustrative embodiments of the invention have been described with particularity, it will be understood that various other modifications will be apparent to and may be readily made by those skilled in the art without departing from the spirit and scope of the invention. Accordingly, it is not intended that the scope of the claims hereof be limited to the examples and descriptions set forth herein but rather that the claims be construed as encompassing all the features of patentable novelty which reside in the present invention, including all features which would be treated as equivalents thereof by those skilled in the art to which the invention pertains.

What is claimed is:

1. A process for preparing polytetramethylene ether glycol comprising the steps of:
(a) contacting a feedstock comprising polytetramethylene ether glycol with an acid ion exchange resin in a contact vessel for a contact time that is sufficient to partially depolymerize the polytetramethylene ether glycol, wherein the acid ion exchange resin has a concentration of acid sites that is greater than about 5 eq/kg, a particle size that is greater than about 0.3 mm, a surface area in the range from about 25 to about 50 m²/gram, and a pore volume in the range from about 0.15 to about 0.35 cc/gram, and wherein the contact vessel has a temperature in the range from about 50 to about 120° C. and a pressure in the range from about 0 to about 30 psig; and
(b) recovering a product stream from the contact vessel comprising a polytetramethylene ether glycol product, wherein the polytetramethylene ether glycol product has an increased number average molecular weight, a lowered polydispersity and a lower molecular weight ratio when compared to the polytetramethylene ether glycol in the feedstock.

2. The process of claim 1 wherein the contact time is in the range from about 5 minutes to about 200 minutes.

3. The process of claim 1 wherein the contact time is in the range from about 5 minutes to about 60 minutes.

4. The process of claim 1 wherein the acid ion exchange resin loses at least a portion of its catalytic properties at temperature above 130° C.

5. The process of claim 1 wherein the acid ion exchange resin loses at least a portion of its catalytic properties at temperature above 150° C.

6. The process of claim 1 wherein the contact vessel is a continuous stirred tank reactor.

7. The process of claim 1 wherein the contact vessel is a fixed bed reactor.

8. The process of claim 1 wherein the temperature is in the range from about 60 to about 100° C. and the pressure is atmospheric pressure.

9. The process of claim 3 wherein the temperature is in the range from about 60 to about 100° C. and pressure is atmospheric pressure.

10. The process of claim 1 wherein the polytetramethylene ether glycol in the feedstock contains less than 1% by weight of polytetrahydrofuran or tetrahydrofuran copolymers.

11. The process of claim 1 wherein polytetramethylene ether glycol in the feedstock contains less than 1% by weight of esters of polytetrahydrofuran or tetrahydrofuran copolymer.

12. The process of claim 1 further comprising recovering an effluent comprising tetrahydrofuran and water from the contact vessel.

13. The process of claim 1 wherein the polytetramethylene ether glycol in the feedstock has a number average molecular weight in the range from about 400 to about 3,000 dalton, a polydispersity in the range from about 1.9 to about 2.3 and a molecular weight ratio in the range from about 2.2 to about 2.5, and wherein the polytetramethylene ether glycol product has a number average molecular weight in the range from about 400 to about 4,000 dalton, a polydispersity in the range from about 1.2 to about 1.8, and a molecular weight ratio in the range from about 1.7 to about 2.15.

14. The process of claim 1 wherein polytetramethylene ether glycol in the feedstock is obtained from the process to covert a diacetate of polymerized tetrahydrofuran to polytetramethylene ether glycol.
